(19) United States
(12) Patent Application Publication
(10) Pub. No.: US 2025/0100965 A1
(43) Pub. Date: Mar. 27, 2025

(54) SEPARATION OF IMPURITIES IN A PROCESS FOR HYDROLYTICALLY DEPOLYMERIZING A POLYAMIDE

(71) Applicant: BASF SE, Ludwigshafen am Rhein (DE)

(72) Inventors: Stefan BLEI, Ludwigshafen am Rhein (DE); Faissal-Ali EL-TOUFAILI, Ludwigshafen am Rhein (DE); Alexander HETZ-HUNSINGER, Ludwigshafen am Rhein (DE); Marc FELDEN, Ludwigshafen am Rhein (DE); Jochen GAUER, Ludwigshafen am Rhein (DE); Michel GASSMANN, Ludwigshafen am Rhein (DE); Christian DIENES, Ludwigshafen am Rhein (DE); Zeljko KOTANJAC, Ludwigshafen am Rhein (DE); Vikram Raghavendhar RAVIKUMAR, Ludwigshafen am Rhein (DE); Esther Matyka LARYEA, Ludwigshafen am Rhein (DE)

(21) Appl. No.: 18/852,720

(22) PCT Filed: Mar. 30, 2023
(86) PCT No.: PCT/EP2023/058281

§ 371 Date: Sep. 30, 2024

(30) Foreign Application Priority Data
Apr. 1, 2022 (EP) 22166283.6

(51) Int. Cl. C07D 201/12; C07D 201/16

(52) U.S. Cl. CPC C07D 201/12; C07D 201/16

(57) ABSTRACT

The present invention relates to a process for separating at least one ε-caprolactam oligomeric compound CPO from a stream S_{R} comprising said at least one CPO and ε-caprolactam monomeric compound CPM.

The present invention relates to a process for separating at least one ε-caprolactam oligomeric compound CPO from a stream comprising said at least one CPO and ε-caprolactam monomeric compound CPM.

Polyamide, and in particular polyamide 6 being characterized by the formula (—NH—(CH₂)₅—CO—)ₙ, can be found in numerous materials, such as packaging, engineering plastics from automotive and textile filaments. The latter represents about 40% of the polyamide 6 global market. At present, only a very small part of the textile filaments is recycled while it represents a significant percentage of the global CO₂ emissions. There is thus a need to recycle polyamide 6 from such materials. Processes for alkaline depolymerizing a polyamide exists. However, such processes have a certain CO₂ footprint and are energy-intensive. Thus, there is a need to provide an improved process for depolymerizing a polyamide able to overcome these issues.

Surprisingly, it was found that the process of the present invention permits to efficiently separate ε-caprolactam oligomers from ε-caprolactam monomeric compounds compared to known processes, which permits among others to improve recycling of solid material comprising a polyamide, such as textile waste materials, in particular after hydrolytical depolymerization of a polyamide. Hence, using a process for separating at least one ε-caprolactam oligomeric compound CPO from a stream SR comprising said at least one CPO and ε-caprolactam monomeric compound CPM according to the present invention permits to reduce the CO₂ footprint.

Therefore, the present invention relates to a process for separating at least one ε-caprolactam oligomeric compound CPO from a stream S_{R} comprising said at least one CPO and ε-caprolactam monomeric compound CPM, the process comprising
(i) providing an aqueous liquid stream S_{R} comprising CPM dissolved in water at a concentration c_{R}(CPM), CPM having a boiling point T_{CPM}, wherein S_{R} further comprises the at least one CPO at a concentration c_{R}(CPO), CPO having a boiling point T_{CPO} with T_{CPO}>T_{CPM};
(ii) preparing an aqueous liquid mixture M_{E} comprising the stream S_{R} provided according to (i);
(iii) subjecting the mixture M_{E} according to (ii) to evaporation conditions in an evaporation unit E₁, obtaining an aqueous vapor stream S_{V1} and an aqueous liquid stream S_{L1}, wherein S_{V1} comprises CPM at a concentration c_{V1}(CPM) with c_{V1}(CPM)>c_{R}(CPM), wherein S_{L1} comprises the at least one CPO at a concentration c_{L1}(CPO) with c_{L1}(CPO)>c_{R}(CPO) and comprises CPM at a concentration c_{L1}(CPM) with c_{L1}(CPM)<c_{R}(CPM);
(iv) dividing the stream S_{L1} according to (iii) into a first stream S_{L11} and a second stream S_{L12}, wherein S_{L11} and S_{L12} have the same chemical composition as S_{L1};
(v) passing the stream S_{L12} obtained according to (iv) to a downstream treatment stage; wherein preparing the aqueous liquid mixture M_{E} according to (ii) comprises mixing the stream S_{R} with the stream S_{L11}.

In the context of the present invention, the term “ε-caprolactam oligomeric compound” (CPO) encompasses any non-monomeric compounds form of ε-caprolactam oligomeric, i.e. any oligomeric compound form of ε-caprolactam including polyamide 6.

Preferably, the stream S_{R} provided according to (i) has a temperature in the range of from 100 to 150° C., more preferably in the range of from 110 to 145° C., more preferably in the range of from 120 to 140° C.

Preferably,
the stream S_{R} provided according to (i) exhibits a CPM concentration in the range of from 15 to 90 weight-%, more preferably in the range of from 20 to 75 weight-%, more preferably in the range of from 25 to 60 weight-%, and a CPO concentration in the range of from 0.5 to 10 weight-%, more preferably in the range of from 0.5 to 7 weight-%, more preferably in the range of from 0.5 to 4 weight-%;
the stream S_{V1} obtained according to (iii) exhibits a CPM concentration in the range of from 65 to 99 weight-%, more preferably in the range of from 65 to 90 weight-%, more preferably in the range of from 65 to 80 weight-%, wherein the stream S_{V1} obtained according to (iii) more preferably exhibits a CPO concentration in the range of from 0 to 0.4 weight-%, more preferably in the range of from 0 to 0.3 weight-%, more preferably in the range of from 0 to 0.2 weight-%;
the stream S_{L1} obtained according to (iii) exhibits a CPM concentration in the range of from 0.1 to 10 weight-%, more preferably in the range of from 0.5 to 7.5 weight-%, more preferably in the range of from 1 to 5 weight-%, and a CPO concentration in the range of from 1 to 10 weight-%, more preferably in the range of from 1.5 to 10 weight-%, more preferably in the range of from 2 to 10 weight-%.

Preferably, according to a first alternative as to the preparation of the aqueous liquid mixture M_{E}, preparing the aqueous liquid mixture M_{E} according to (ii) further comprises, prior to mixing the stream S_{R} with the stream S_{L11}, heating the stream S_{L11} obtained from (iv) to a temperature in the range of from 200 to 270° C., more preferably in the range of from 210 to 270° C., more preferably in the range of from 220 to 270° C.

Preferably, according to said first alternative, heating the stream S_{L11} comprises passing the stream S_{L11} through a heat exchanger H₁. Preferably, according to said first alternative, according to (ii), the stream S_{R} provided according to (i) and the stream S_{L11} obtained from heating are mixed in the evaporation unit E₁. Preferably, according to said first alternative, prior to mixing the stream S_{R} with the stream S_{L11}, the stream S_{R} provided according to (i) is not heated.

It is conceivable according to said first alternative that one or more heat exchangers upstream of H₁ be preferably used. Preferably, no further heat exchanger is used upstream of H₁.

The first alternative as to the preparation of the aqueous liquid mixture M_{E} is in particular illustrated by FIG. 1.

Preferably, according to a second alternative as to the preparation of the aqueous liquid mixture M_{E}, preparing the aqueous liquid mixture M_{E} according to (ii) comprises mixing the stream S_{R} provided according to (i) with the stream S_{L11} obtained from (iv) and heating the combined stream to a temperature in the range of from 200 to 270° C., more preferably in the range of from 210 to 270° C., more preferably in the range of from 220 to 270° C.

Preferably heating the combined stream comprises passing the stream S_{L11} through a heat exchanger H₁. Preferably, prior to mixing the stream S_{R} with the stream S_{L11}, the stream S_{R} provided according to (i) is not heated. It is conceivable according to said second alternative that one or more heat exchangers upstream of H₁ be preferably used. Preferably, no further heat exchanger is used upstream of H₁.

The first alternative as to the preparation of the aqueous liquid mixture M_{E} is in particular illustrated by FIG. 2.

In the context of the present invention, preferably, the evaporation in the evaporation unit E₁ according to (iii) is carried out in one or more stirred vessels, or in one or more film evaporators, or in one or more stirred vessels and in one or more film evaporators. More preferably, the evaporation in the evaporation unit E₁ according to (iii) is carried out in one or more continuous stirred-tank reactors, or in one or more falling film evaporators, or in one or more continuous stirred-tank reactors and in one or more falling film evaporators. More preferably, the evaporation in the evaporation unit E₁ according to (iii) is carried out in one or more continuous stirred-tank reactors, wherein more preferably, if evaporation in E₁ is carried out in more than one continuous stirred-tank reactors, the continuous stirred-tank reactors are arranged in parallel.

Preferably, the one or more stirred vessels and the one or more film evaporators are equipped with heating means to indirectly providing heat for the evaporation carried out in E₁, the process comprising passing a heating medium through said heating means, wherein said heating means are more preferably heating jackets.

Preferably, the evaporation conditions according to (iii) comprise an evaporation temperature T_{E1} of the mixture M_{E}, wherein T_{E1} is in the range of from 200 to 270° C., more preferably in the range of from 210 to 270° C., more preferably in the range of from 220 to 270° C., and the evaporation conditions according to (iii) further comprise an evaporation pressure p_{E1}, wherein p_{E1} is more preferably less than 1 bar(abs).

Preferably, p_{E1} is in the range of from 10 to 900 mbar(abs), more preferably in the range of from 10 to 850 mbar(abs), more preferably in the range of from 10 to 800 mbar(abs).

Preferably, the evaporation conditions according to (iii) further comprise a residence time t_{E1} in the evaporation unit E₁, wherein t_{E1} is in the range of from 1 min to 5 h, more preferably in the range of from 5 min to 4 h, more preferably in the range of from 10 min to 3 h. More preferably, the evaporation conditions according to (iii) comprise an evaporation temperature T_{E1} of the mixture M_{E}, wherein T_{E1} is in the range of from 200 to 270° C., more preferably in the range of from 210 to 270° C., more preferably in the range of from 220 to 270° C.; the evaporation conditions according to (iii) further comprise an evaporation pressure p_{E1}, wherein p_{E1} is more preferably less than 1 bar(abs), more preferably in the range of from 10 to 900 mbar(abs), more preferably in the range of from 10 to 850 mbar(abs), more preferably in the range of from 10 to 800 mbar(abs); and the evaporation conditions according to (iii) further comprise a residence time t_{E1} in the evaporation unit E₁, wherein t_{E1} is in the range of from 1 min to 5 h, more preferably in the range of from 5 min to 4 h, more preferably in the range of from 10 min to 3 h.

Preferably, according to (iv), the stream S_{L1} is divided into the first stream S_{L11} and the second stream S_{L12} at a mass ratio m(S_{L12}):m(S_{L11}) in the range of from 0.01:1 to 0.02:1.

Preferably, the downstream treatment stage according to (v) comprises one or more of
an evaporation unit;
a depolymerization unit for depolymerizing at least one of the at least one ε-caprolactam oligomeric compound CPO comprised in the stream S_{L12};
a separation unit for separating at least one solid residue from the stream S_{L12};
a processing unit for processing at least one solid residue comprised in the stream S_{L12};
an incineration stage for incinerating at least one solid residue comprised in the stream S_{L12}.

Preferably, the downstream treatment stage according to (v) comprises an evaporation unit and the process further comprises
(vi) subjecting the stream S_{L12} to evaporation conditions in an evaporation unit E₂, obtaining an aqueous vapor stream S_{V2} and a liquid stream S_{L2} wherein S_{V2} comprises CPM at a concentration c_{V2}(CPM) with c_{V2}(CPM)>c_{L12}(CPM), and CPO at a concentration c_{V2}(CPO) with c_{V2}(CPO)<c_{L12}(CPO); wherein S_{L2} comprises the at least one CPO at a concentration c_{L2}(CPO) with c_{L2}(CPO)≥c_{L12}(CPO);
(vii) passing the stream S_{L2} obtained according to (vi) to a downstream treatment stage.

The liquid stream S_{L2} is preferably a non-aqueous liquid stream, i.e. a liquid stream having a water content of at most 0.01 weight-%, preferably of at most 0.005 weight-%, preferably of most 0.002 weight-%, more preferably of at most 0.001 weight-%.

Preferably,
the stream S_{V2} obtained according to (vi) exhibits a CPM concentration in the range of from 50 to 100 weight-%, more preferably in the range of from 60 to 100 weight-%, more preferably in the range of from 80 to 100 weight-%, and a CPO concentration in the range of from 0 to 0.5 weight-%, more preferably in the range of from 0 to 0.3 weight-%, more preferably in the range of from 0 to 0.1 weight-%;
the stream S_{L2} obtained according to (vi) exhibits a CPO concentration in the range of from 1 to 10 weight-%, more preferably in the range of from 1.5 to 10 weight-%, more preferably in the range of from 2 to 10 weight-%.

Preferably the evaporation in the evaporation unit E₂ according to (vi) is carried out in one or more stirred vessels, or in one or more film evaporators, or in one or more stirred vessels and in one or more film evaporators. More preferably the evaporation in the evaporation unit E₂ according to (vi) is carried out in one or more film evaporators. More preferably the evaporation in the evaporation unit E₂ according to (vi) is carried out in one or more wipe film evaporators, wherein more preferably, if evaporation in E₂ is carried out in more than one wipe film evaporators, the wipe film evaporators are arranged in parallel.

It is conceivable that the evaporation in the evaporation unit E₂ according to (vi) be carried out in one or more kneaders.

Preferably the one or more stirred vessels and the one or more film evaporators are equipped with heating means to indirectly providing heat for the evaporation carried out in E₂, the process comprising passing a heating medium through said heating means, wherein said heating means are more preferably heating jackets.

Preferably the evaporation conditions according to (vi) comprise an evaporation temperature T_{E2} of the stream S_{L12}, wherein T_{E2} is in the range of from 200 to 300° C., more preferably in the range of from 215 to 300° C., more preferably in the range of from 230 to 300° C., and the evaporation conditions according to (vi) further comprise an evaporation pressure p_{E2}, wherein p_{E2} is more preferably less than 1 bar(abs).

Preferably, p_{E2} is in the range of from 10 to 900 mbar(abs), more preferably in the range of from 10 to 850 mbar(abs), more preferably in the range of from 10 to 800 mbar(abs).

Preferably, the evaporation conditions according to (vi) further comprise a residence time t_{E2} in the evaporation unit E₂, wherein t_{E2} is in the range of from 1 s to 5 min, more preferably in the range of from 5 s to 4 min, more preferably in the range of from 10 s to 3 min. More preferably, the evaporation conditions according to (vi) comprise an evaporation temperature T_{E2} of the stream S_{L12}, wherein T_{E2} is in the range of from 200 to 300° C., more preferably in the range of from 215 to 300° C., more preferably in the range of from 230 to 300° C.; the evaporation conditions according to (vi) further comprise an evaporation pressure p_{E2}, wherein p_{E2} is more preferably less than 1 bar(abs), more preferably in the range of from 10 to 900 mbar(abs), more preferably in the range of from 10 to 850 mbar(abs), more preferably in the range of from 10 to 800 mbar(abs); and the evaporation conditions according to (vi) further comprise a residence time t_{E2} in the evaporation unit E₂, wherein t_{E2} is in the range of from 1 s to 5 min, more preferably in the range of from 5 s to 4 min, more preferably in the range of from 10 s to 3 min.

Preferably, the downstream treatment stage according to (vii) comprises one or more of
a depolymerization unit for depolymerizing at least one of the at least one ε-caprolactam oligomeric compound CPO comprised in the stream S_{L2};
a separation unit for separating at least one solid residue from the stream S_{L2};
a processing unit for processing at least one solid residue comprised in the stream S_{L2};
an incineration stage for incinerating at least one solid residue comprised in the stream S_{L2}.

Preferably, the process further comprises
(viii) passing the aqueous vapor stream S_{V1}, more preferably the aqueous vapor stream S_{V1} and the aqueous vapor stream S_{V2}, more preferably a combined stream of the aqueous vapor stream S_{V1} and the aqueous vapor stream S_{V2}, to a water removal unit W_{U} for separating CPM from water, wherein the water removal unit more preferably comprises at least one distillation column, more preferably from 1 to 3 distillation columns, more preferably 2 or 3 distillation columns, more preferably 3 distillation columns, wherein, if the water removal unit W_{U} comprises more than one distillation column, the distillation columns are more preferably serially arranged.

Preferably, the bottoms stream of at least one distillation column, more preferably the bottoms stream of the downstream-most distillation column, is recycled into the evaporation unit E₁.

Preferably providing the stream S_{R} according to (i) comprises
(i.1) preparing an aqueous liquid mixture M_{WC} containing CPM dissolved in water and CPO, comprising
   (i.1.1) providing an aqueous liquid stream S_{W};
   (i.1.2) providing a solid material M containing a polyamide prepared from ε-caprolactam;
   (i.1.3) preparing a mixture of the solid material M provided according to (i.1.2) and the aqueous liquid stream S_{W} provided according to (i.1.1);
   (i.1.4) preparing an aqueous liquid mixture M_{WP} comprising the polyamide dissolved in water from the mixture prepared according to (i.1.3);
   (i.1.5) subjecting the aqueous liquid mixture M_{WP} prepared according to (i.1.4) to depolymerization conditions in a chemical reactor unit R_{U}, obtaining the aqueous liquid mixture M_{WC} comprising CPM dissolved in water and CPO;
(i.2) optionally subjecting the aqueous mixture M_{WC} obtained according to (i.1.5) to depressurization in a depressurization unit D_{U}, obtaining an aqueous vapor stream S_{VD} and an aqueous liquid stream S_{C}, S_{C} comprising CPM dissolved in water and CP;
(i.3) optionally passing the aqueous mixture M_{WC} obtained according to (i.1.5) or the aqueous liquid stream S_{C}, obtained according to (i.2) to solid-liquid separation in a solid-liquid separation unit SL_{U}, obtaining an aqueous liquid stream S_{L} comprising CPM dissolved in water and CPO;
(i.4) separating water from the aqueous liquid mixture M_{WC} obtained according to (i.1.5), or from the aqueous liquid stream S_{C} obtained according to (i.2), or from the aqueous liquid stream S_{L} obtained according to (i.3), by evaporation in an evaporation unit E_{U} comprising least two evaporation sub-units E_{U1} and E_{U2}, obtaining at least one aqueous vapor stream S_{VE} and the aqueous liquid stream S_{R}.

Preferably the solid material M provided according to (i.1.2) comprises, more preferably consists of waste material, wherein said waste material more preferably comprises textile waste material.

Preferably, preparing an aqueous liquid stream S_{WC} containing ε-caprolactam dissolved in water according to (i.1) comprises
(i.1.1) providing an aqueous liquid stream S_{W}, wherein from 50 weight-% to 100 weight-% of S_{W} consist of water and wherein S_{W} has a temperature T_{SW}, wherein T_{SW}>T_{P};
(i.1.2) providing a solid material M containing the polyamide prepared from ε-caprolactam, M having a temperature T_{M}, wherein T_{M}<T_{P}, T_{P} being the melting point of the polyamide;
(i.1.3) preparing an aqueous mixture of the solid material M provided according to (i.1.2) and the aqueous liquid stream S_{W} provided according to (i.1.1), comprising feeding the solid material M provided according to (i.1.2) and the liquid aqueous stream S_{W} provided according to (i.1.1) into a chemical reactor unit R_{U} obtaining said mixture;
(i.1.4) preparing an aqueous liquid mixture M_{WP} comprising the polyamide dissolved in water from the mixture prepared according to (i.1.3), comprising feeding the solid material M provided according to (i.1.2) and the liquid aqueous stream S_{W} provided according to (i.1.1) into a chemical reactor unit R_{U} obtaining said mixture;
(i.1.5) subjecting the aqueous liquid mixture prepared according to (i.1.4) to depolymerization conditions in a chemical reactor unit R_{U}, obtaining the aqueous liquid stream M_{WC} containing ε-caprolactam dissolved in water, wherein the depolymerization conditions comprise a depolymerization temperature T_{D} at a depolymerization pressure p_{D}, wherein T_{M}<T_{D}<T_{SW}.

Preferably, T_{D} is in the range of from 230 to 320° C., more preferably in the range of from 250 to 300° C., more preferably in the range of from 250 to 295° C. or from 270 to 295° C.

Preferably, T_{SW} is in the range of from 250 to 350° C., more preferably in the range of from 260 to 330° C., more preferably in the range of from 290 to 325° C.

The excess heat from the liquid aqueous stream S_{W} (T_{SW}) melts the solid material M containing the polyamide, the solid material M preferably being in the form of granules, and provides the needed reaction enthalpy. Any additional heat required to maintain the reaction temperature can be provided through a heating jacket using hot oil of the reactor unit R_{U}. This is detailed in the following.

Preferably, ΔT=T_{SW}-T_{P} is in the range of from 10 to 70° C., more preferably in the range of from 10 to 50° C., more preferably in the range of from 10 to 30° C.

Preferably, p_{D} is in the range of from 40 to 120 bar, more preferably in the range of from 50 to 100 bar, more preferably in the range of from 60 to 90 bar.

Preferably, in the mixture prepared according to (i.1.3), at least 75 weight-%, more preferably from 75 to 100 weight-%, more preferably from 85 to 100 weight-%, more preferably from 95 to 100 weight-%, of the polyamide comprised in the solid material M are comprised in liquid form.

Preferably, from 91 to 100 weight-%, more preferably from 92 to 100 weight-%, more preferably from 95 to 100 weight-% of S_{W} provided according to (i.1.1) consist of water.

Preferably, the solid material M provided according to (i.1.2) comprises, more preferably consists of waste material, wherein said waste material more preferably comprises textile waste material.

Preferably from 10 to 99 weight-%, more preferably from 30 to 98.5 weight-%, more preferably from 50 to 98 weight-%, more preferably from 80 to 98 weight-%, of M consist of the polyamide; or preferably from 10 to 100 weight-%, more preferably from 30 to 100 weight-%, more preferably from 50 to 100 weight-%, more preferably from 80 to 100 weight-%, of M consist of the polyamide.

Preferably, M is in the form of granules, wherein the mean diameter of the granules is more preferably in the range of from 0.5 to 10 mm, more preferably in the range of from 1 to 7 mm, more preferably in the range of from 2 to 4 mm.

According to (i.1.4), M and S_{W} are preferably fed into R_{U} at a mixing ratio m_{W}/kg:m_{P}/kg, defined as the amount of water contained in S₁, m_{W}, relative to the mass of polyamide contained in M, m_{P}, in the range of from 1:1 to 20:1, more preferably in the range of from 2:1 to 15:1, more preferably in the range of from 5:1 to 10:1.

Preferably, the chemical reactor unit R_{U} comprises z chemical reactors Rᵢ=1 . . . z, wherein z is in the range of from 1 to 10, more preferably in the range of from 2 to 8, more preferably in the range of from 2 to 6, more preferably in the range of from 2 to 5, more preferably in the range of from 2 to 4, more preferably 3 or 4, more preferably 4.

Preferably, z>1 and at least two reactors Rᵢ, more preferably the z reactors Rᵢ are serially coupled.

Preferably, the z reactors Rᵢ are serially coupled, wherein
according to (i.1.4), M_{WP} is fed into Rᵢ, i=1;
an aqueous liquid stream Sᵢ containing ε-caprolactam dissolved in water is removed from Rᵢ and fed into Rᵢ₊₁, i<z;
according to (i.1.5), M_{WC} is removed from R_{z};

wherein in every reactor Rᵢ, a depolymerization temperature T_{Di} at a depolymerization pressure p_{Di} are maintained, wherein, independently of each other, T_{Di} is in the range of from 230 to 320° C., more preferably in the range of from 250 to 300° C., more preferably in the range of from 270 to 295° C., and wherein, independently of each other, p_{Di} is more preferably in the range of from 40 to 120 bar, more preferably in the range of from 50 to 100 bar, more preferably in the range of from 60 to 90 bar.

Preferably maintaining a depolymerization temperature T_{Di} in a reactor Rᵢ comprises heating the reactor contents of Rᵢ, more preferably indirectly heating the reactor contents of Rᵢ, wherein more preferably, maintaining a depolymerization temperature T_{Di} in a reactor Rᵢ comprises heating the reactor contents of Rᵢ by passing a heating medium through a heating jacket of Rᵢ. The heating medium is preferably hot oil. However, it is noted that other heating medium known by the skilled person can be used for passing through the heating jacket of Rᵢ.

Preferably, the z reactors Rᵢ are vertically arranged, with R₁ being the top-most reactor and R_{z} being the bottom-most reactor, wherein Sᵢ obtained from Rᵢ is transferred to Rᵢ₊₁ by gravity, more preferably by gravity only.

Preferably, at least 1, more preferably z reactors Rᵢ, are configured as non-stirred reactors or as non-circulation reactors, more preferably as non-stirred reactors and non-circulation reactors. The system is preferably operated without stirrer and without circulation pump. That avoids difficult sealings, i.e. high pressure sealings, for the stirrer and pump.

As mentioned in the foregoing, the mixing is preferably ensured by dosing the amount of water stepwise with ongoing reaction time, and dosing the liquid solution, for example, from reactor R₁ by gravity to reactor R₂ and R₃ after parts of the reaction time.

Preferably, the reactors R₁ to R_{y-1} are operated in batch mode and the reactors R_{y} to R_{z} are operated in continuous mode, wherein y>1 and y≤z, wherein y is more preferably z.

Preferably, the residence time in one or more of the reactors R₁ to R_{y-1}, more preferably in the reactors R₁ to R_{y-1}, is in the range of from 5 to 40 minutes, more preferably in the range of from 10 to 30 minutes, more preferably in the range of from 15 to 25 minutes.

Preferably, the residence time in the reactor R_{y}, wherein y is more preferably z, is in the range of from 1 second to 40 minutes, more preferably in the range of from 2 seconds to 30 minutes, more preferably in the range of from 3 seconds to 25 minutes.

Preferably, the overall residence time in the chemical reactor unit is in the range of from 15 to 160 minutes, more preferably in the range of from 30 to 120 minutes, more preferably in the range of from 45 to 100 minutes, more preferably in the range of from 60 to 80 minutes.

More preferably, four identical chemical reactors R₁-R₄ are arranged in series. The first three reactors, R₁-R₃, are preferably operated in batch mode, all of said reactors having low residence time and the last, R₄, in continuous mode to enable continuous feeding of the downstream process steps.

As to the feeding of S_{W} and M, it is preferred that M and S_{W} are fed into Rᵢ one after the other. More preferably, S_{W} is fed into Rᵢ and subsequently M is fed into Rᵢ containing S_{W}. After a time T, Sᵢ is removed from Rᵢ and fed to Rᵢ₊₁.

Preferably, subjecting the aqueous mixture M_{WC} obtained according to (i.1.5) to depressurization in a depressurization unit D_{U} according to (i.2) comprises
(i.2.1) feeding the aqueous liquid stream M_{WC} as a feed stream to a first evaporation sub-unit DU11, obtaining an aqueous vapor stream S_{VD11}, and an aqueous liquid stream S_{L11} comprising ε-caprolactam dissolved in water; the aqueous liquid stream M_{WC} is optionally passed through at least one solid-liquid separation unit F1;
(i.2.2) feeding the aqueous liquid stream S_{L11} as a feed stream to a second evaporation sub-unit DU12, obtaining an aqueous vapor stream S_{VD12}, and the aqueous liquid stream S_{C} comprising ε-caprolactam dissolved in water, the aqueous liquid stream S_{L11} is optionally passed through at least one solid-liquid separation unit F2;
wherein (ii.1) comprises at least one of passing M_{WC} through F1 and passing S_{L11} through F2,
wherein (ii.1) more preferably comprises passing M_{WC} through F1 and passing S_{L11} through F2. This is in particular illustrated in FIG. 8. The combination of S_{VD11} and S_{VD12} corresponds to S_{VD}.

Preferably, at least one of the solid-liquid separation unit F1 and the solid-liquid separation unit F2, more preferably the solid-liquid separation unit F1 and the solid-liquid separation unit F2 are filtration units, wherein F1 more preferably has a mesh size in the range of from 0.5 to 5 mm, more preferably in the range of from 1 to 3 mm, and F2 more preferably has a mesh size in the range of from 0.5 to 5 mm, more preferably in the range of from 1 to 3 mm.

Preferably, according to (i.4), the at least two evaporation sub-units E_{U1} and E_{U2} are serially coupled, wherein an aqueous vapor stream S_{VE1} is obtained from E_{U1} and an aqueous vapor stream S_{VE2} is obtained from E_{U2}, and wherein from E_{U2}, an aqueous liquid stream S_{R} comprising ε-caprolactam dissolved in water is obtained; more preferably the evaporation unit E_{U} comprises two evaporation units E_{U1} and E_{U2},
wherein preferably from 75 to 100 weight-% of the aqueous liquid stream which is fed to evaporation according to (i.4) consist of water and ε-caprolactam, said stream exhibiting a water concentration c_{H2O} and preferably having a concentration of ε-caprolactam c_{CPL} in the range of from 5 to 20 weight-%; the process further comprising recycling at least a part of at least one of streams S_{VE1} and S_{VE2} as a component of the aqueous liquid stream S_{W}, said recycling preferably comprising condensing the at least one of streams S_{VE1} and S_{VE2}. The combination of S_{VE1} and S_{VE2} corresponds to S_{VE}.

Preferably, according to (vii), passing the liquid stream S_{L2} to a downstream treatment stage comprises
(vii.1) dividing the stream S_{L2} into a first stream S_{L21} and a second stream S_{L22}, wherein S_{L21} and S_{L22} have the same chemical composition as S_{L2};
(vii.2) recycling the stream S_{L21} into the chemical reactor unit R_{U} according to (i.1.5);
(vii.3) passing the stream S_{L22} to a downstream treatment stage.

Preferably, the downstream treatment stage according to (vii.3) comprises one or more of
a separation unit for separating at least one solid residue from the stream S_{L22};
a processing unit for processing at least one solid residue comprised in the stream S_{L22};
an incineration stage for incinerating at least one solid residue comprised in the stream S_{L22}.

The present invention is further illustrated by the following set of embodiments and combinations of embodiments resulting from the dependencies and back-references as indicated. In particular, it is noted that in each instance where a range of embodiments is mentioned, for example in the context of a term such as “The process of any one of embodiments 1 to 3”, every embodiment in this range is meant to be explicitly disclosed for the skilled person, i.e. the wording of this term is to be understood by the skilled person as being synonymous to “The process of any one of embodiments 1, 2 and 3”. Further, it is explicitly noted that the following set of embodiments represents a suitably structured part of the general description directed to preferred aspects of the present invention, and, thus, suitably supports, but does not represent the claims of the present invention.
1. A process for separating at least one ε-caprolactam oligomeric compound CPO from a stream S_{R} comprising said at least one CPO and ε-caprolactam monomeric compound CPM, the process comprising
   (i) providing an aqueous liquid stream S_{R} comprising CPM dissolved in water at a concentration c_{R}(CPM), CPM having a boiling point T_{CPM}, wherein S_{R} further comprises the at least one CPO at a concentration c_{R}(CPO), CPO having a boiling point T_{CPO} with T_{CPO}>T_{CPM};
   (ii) preparing an aqueous liquid mixture M_{E} comprising the stream S_{R} provided according to (i);
   (iii) subjecting the mixture M_{E} according to (i) to evaporation conditions in an evaporation unit E₁, obtaining an aqueous vapor stream S_{V1} and an aqueous liquid stream S_{L1}, wherein S_{V1} comprises CPM at a concentration c_{V1}(CPM) with c_{V1}(CPM)>c_{R}(CPM), wherein S_{L1} comprises the at least one CPO at a concentration c_{L1}(CPO) with c_{L1}(CPO)>c_{R}(CPO) and comprises CPM at a concentration c_{L1}(CPM) with c_{L1}(CPM)<c_{R}(CPM);
   (iv) dividing the stream S_{L1} according to (iii) into a first stream S_{L11} and a second stream S_{L12}, wherein S_{L11} and S_{L12} have the same chemical composition as S_{L1};
   (v) passing the stream S_{L12} obtained according to (iv) to a downstream treatment stage; wherein preparing the aqueous liquid mixture M_{E} according to (ii) comprises mixing the stream S_{R} with the stream S_{L11}.
2. The process of embodiment 1, wherein the stream S_{R} provided according to (i) has a temperature in the range of from 100 to 150° C., preferably in the range of from 110 to 145° C., more preferably in the range of from 120 to 140° C.
3. The process of embodiment 1 or 2,
   wherein the stream S_{R} provided according to (i) exhibits a CPM concentration in the range of from 15 to 90 weight-%, preferably in the range of from 20 to 75 weight-%, more preferably in the range of from 25 to 60 weight-%; and a CPO concentration in the range of from 0.5 to 10 weight-%, preferably in the range of from 0.5 to 7 weight-%, more preferably in the range of from 0.5 to 4 weight-%;
   wherein the stream S_{V1} obtained according to (iii) exhibits a CPM concentration in the range of from 65 to 99 weight-%, preferably in the range of from 65 to 90 weight-%, more preferably in the range of from 65 to 80 weight-%; wherein the stream S_{V1} obtained according to (iii) preferably exhibits a CPO concentration in the range of from 0 to 0.4 weight-%, preferably in the range of from 0 to 0.3 weight-%, more preferably in the range of from 0 to 0.2 weight-%;
   wherein the stream S_{L1} obtained according to (iii) exhibits a CPM concentration in the range of from 0.1 to 10 weight-%, preferably in the range of from 0.5 to 7.5 weight-%, more preferably in the range of from 1 to 5 weight-%; and a CPO concentration in the range of from 1 to 10 weight-%, preferably in the range of from 1.5 to 10 weight-%, more preferably in the range of from 2 to 10 weight-%.
4. The process of any one of embodiments 1 to 3, wherein preparing the aqueous liquid mixture M_{E} according to (ii) further comprises, prior to mixing the stream S_{R} with the stream S_{L11}, heating the stream S_{L11} obtained from (iv) to a temperature in the range of from 200 to 270° C., preferably in the range of from 210 to 270° C., more preferably in the range of from 220 to 270° C.
5. The process of embodiment 4, wherein heating the stream S_{L11} comprises passing the stream S_{L11} through a heat exchanger H₁.
6. The process of embodiment 4 or 5, wherein according to (ii), the stream S_{R} provided according to (i) and the stream S_{L11} obtained from heating are mixed in the evaporation unit E₁.
7. The process of any one of embodiments 4 to 6, wherein prior to mixing the stream S_{R} with the stream S_{L11}, the stream S_{R} provided according to (i) is not heated.
8. The process of any one of embodiments 1 to 3, wherein preparing the aqueous liquid mixture M_{E} according to (ii) comprises mixing the stream S_{R} provided according to (i) with the stream S_{L11} obtained from (iv) and heating the combined stream to a temperature in the range of from 200 to 270° C., preferably in the range of from 210 to 270° C., more preferably in the range of from 220 to 270° C.
9. The process of embodiment 8, wherein heating the combined stream comprises passing the stream S_{L11} through a heat exchanger H₁.
10. The process of embodiment 8 or 9, wherein prior to mixing the stream S_{R} with the stream S_{L11}, the stream S_{R} provided according to (i) is not heated.
11. The process of any one of embodiments 1 to 10, wherein the evaporation in the evaporation unit E₁ according to (iii) is carried out in one or more stirred vessels, or in one or more film evaporators, or in one or more stirred vessels and in one or more film evaporators; wherein the evaporation in the evaporation unit E₁ according to (iii) is preferably carried out in one or more continuous stirred-tank reactors, or in one or more falling film evaporators, or in one or more continuous stirred-tank reactors and in one or more falling film evaporators; wherein the evaporation in the evaporation unit E₁ according to (iii) is more preferably carried out in one or more continuous stirred-tank reactors, wherein more preferably, if evaporation in E₁ is carried out in more than one continuous stirred-tank reactors, the continuous stirred-tank reactor are arranged in parallel.
12. The process of embodiment 11, wherein the one or more stirred vessels and the one or more film evaporators are equipped with heating means to indirectly providing heat for the evaporation carried out in E₁, the process comprising passing a heating medium through said heating means, wherein said heating means are preferably heating jackets.
13. The process of any one of embodiments 1 to 12, preferably of embodiment 11 or 12, wherein the evaporation conditions according to (iii) comprise an evaporation temperature T_{E1} of the mixture M_{E}, wherein T_{E1} is in the range of from 200 to 270° C., preferably in the range of from 210 to 270° C., more preferably in the range of from 220 to 270° C., and wherein the evaporation conditions according to (iii) further comprise an evaporation pressure p_{E1}, wherein p_{E1} is preferably less than 1 bar(abs).
14. The process of embodiment 13, wherein p_{E1} is in the range of from 10 to 900 mbar(abs), preferably in the range of from 10 to 850 mbar(abs), more preferably in the range of from 10 to 800 mbar(abs).
15. The process of any one of embodiments 1 to 14, preferably of any one of embodiments 11 to 14, wherein the evaporation conditions according to (iii) further comprise a residence time t_{E1} in the evaporation unit E₁, wherein t_{E1} is in the range of from 1 min to 5 h, preferably in the range of from 5 min to 4 h, more preferably in the range of from 10 min to 3 h.
16. The process of any one of embodiments 1 to 15, wherein according to (iv), the stream S_{L1} is divided into the first stream S_{L11} and the second stream S_{L12} at a mass ratio m(S_{L12}):m(S_{L11}) in the range of from 0.01:1 to 0.02:1.
17. The process of any one of embodiments 1 to 16, wherein the downstream treatment stage according to (v) comprises one or more of
   an evaporation unit;
   a depolymerization unit for depolymerizing at least one of the at least one ε-caprolactam oligomeric compound CPO comprised in the stream S_{L12};
   a separation unit for separating at least one solid residue from the stream S_{L12};
   a processing unit for processing at least one solid residue comprised in the stream S_{L12},
   an incineration stage for incinerating at least one solid residue comprised in the stream S_{L12}.
18. The process of any one of embodiments 1 to 17, wherein the downstream treatment stage according to (v) comprises an evaporation unit, the process further comprising
   (vi) subjecting the stream S_{L12} to evaporation conditions in an evaporation unit E₂, obtaining an aqueous vapor stream S_{V2} and a liquid stream S_{L2} wherein S_{V2} comprises CPM at a concentration c_{V2}(CPM) with c_{V2}(CPM)>c_{L12}(CPM), and CPO at a concentration c_{V2}(CPO) with c_{V2}(CPO)<c_{L12}(CPO); wherein S_{L2} comprises the at least one CPO at a concentration c_{L2}(CPO) with c_{L2}(CPO)≥c_{L12}(CPO);
   (vii) passing the stream S_{L2} obtained according to (vi) to a downstream treatment stage.
19. The process of embodiment 18,
   wherein the stream S_{V2} obtained according to (vi) exhibits a CPM concentration in the range of from 50 to 100 weight-%, preferably in the range of from 60 to 100 weight-%, more preferably in the range of from 80 to 100 weight-%; and a CPO concentration in the range of from 0 to 0.5 weight-%, preferably in the range of from 0 to 0.3 weight-%, more preferably in the range of from 0 to 0.1 weight-%;
   wherein the stream S_{L2} obtained according to (vi) exhibits a CPO concentration in the range of from 1 to 10 weight-%, preferably in the range of from 1.5 to 10 weight-%, more preferably in the range of from 2 to 10 weight-%.
20. The process of embodiment 18 or 19, wherein the evaporation in the evaporation unit E₂ according to (vi) is carried out in one or more stirred vessels, or in one or more film evaporators, or in one or more stirred vessels and in one or more film evaporators; wherein the evaporation in the evaporation unit E₂ according to (vi) is preferably carried out in one or more film evaporators; wherein the evaporation in the evaporation unit E₂ according to (vi) is more preferably carried out in one or more wipe film evaporators, wherein more preferably, if evaporation in E₂ is carried out in more than one wipe film evaporators, the wipe film evaporators are arranged in parallel.
21. The process of embodiment 20, wherein the one or more stirred vessels and the one or more film evaporators are equipped with heating means to indirectly providing heat for the evaporation carried out in E₂, the process comprising passing a heating medium through said heating means, wherein said heating means are preferably heating jackets.
22. The process of any one of embodiments 18 to 21, wherein the evaporation conditions according to (vi) comprise an evaporation temperature T_{E2} of the stream S_{L12}, wherein T_{E2} is in the range of from 200 to 300° C., preferably in the range of from 215 to 300° C., more preferably in the range of from 230 to 300° C., and wherein the evaporation conditions according to (vi) further comprise an evaporation pressure p_{E2}, wherein p_{E2} is preferably less than 1 bar(abs).
23. The process of embodiment 22, wherein p_{E2} is in the range of from 10 to 900 mbar(abs), preferably in the range of from 10 to 850 mbar(abs), more preferably in the range of from 10 to 800 mbar(abs).
24. The process of any one of embodiments 18 to 23, wherein the evaporation conditions according to (vi) further comprise a residence time t_{E2} in the evaporation unit E₂, wherein t_{E2} is in the range of from 1 s to 5 min, preferably in the range of from 5 s to 4 min, more preferably in the range of from 10 s to 3 min.
25. The process of any one of embodiments 18 to 24, wherein the downstream treatment stage according to (vii) comprises one or more of
   a depolymerization unit for depolymerizing at least one of the at least one ε-caprolactam oligomeric compound CPO comprised in the stream S_{L2};
   a separation unit for separating at least one solid residue from the stream S_{L2};
   a processing unit for processing at least one solid residue comprised in the stream S_{L2};
   an incineration stage for incinerating at least one solid residue comprised in the stream S_{L2}.
26. The process of any one of embodiments 1 to 25, preferably of any one of embodiments 18 to 25, the process further comprising
   (viii) passing the aqueous vapor stream S_{V1}, preferably the aqueous vapor stream S_{V1} and the aqueous vapor stream S_{V2}, more preferably a combined stream of the aqueous vapor stream S_{V1} and the aqueous vapor stream S_{V2}, to a water removal unit W_{U} for separating CPM from water, wherein the water removal unit preferably comprises at least one distillation column, more preferably from 1 to 3 distillation columns, more preferably 2 or 3 distillation columns, more preferably 3 distillation columns, wherein, if the water removal unit W_{U} comprises more than one distillation column, the distillation columns are preferably serially arranged.
27. The process of embodiment 26, wherein the bottoms stream of at least one distillation column, preferably the bottoms stream of the downstream-most distillation column is recycled into the evaporation unit E₁.
28. The process of any one of embodiments 1 to 27, wherein providing the stream S_{R} according to (i) comprises
   (i.1) preparing an aqueous liquid mixture M_{WC} containing CPM dissolved in water and CPO, comprising
      (i.1.1) providing an aqueous liquid stream S_{W};
      (i.1.2) providing a solid material M containing a polyamide prepared from ε-caprolactam;
      (i.1.3) preparing a mixture of the solid material M provided according to (i.1.2) and the aqueous liquid stream S_{W} provided according to (i.1.1);
      (i.1.4) preparing an aqueous liquid mixture M_{WP} comprising the polyamide dissolved in water from the mixture prepared according to (i.1.3);
      (i.1.5) subjecting the aqueous liquid mixture M_{WP} prepared according to (i.1.4) to depolymerization conditions in a chemical reactor unit R_{U}, obtaining the aqueous liquid mixture M_{WC} comprising CPM dissolved in water and CPO;
   (i.2) optionally subjecting the aqueous mixture M_{WC} obtained according to (i.1.5) to depressurization in a depressurization unit D_{U}, obtaining an aqueous vapor stream S_{VD} and an aqueous liquid stream S_{C}, S_{C} comprising CPM dissolved in water and CPO;
   (i.3) optionally passing the aqueous mixture M_{WC} obtained according to (i.1.5) or the aqueous liquid stream S_{C}, obtained according to (i.2) to solid-liquid separation in a solid-liquid separation unit SL_{U}, obtaining an aqueous liquid stream S_{L} comprising CPM dissolved in water and CPO;
(i.4) separating water from the aqueous liquid mixture M_{WC} obtained according to (i.1.5) or from the aqueous liquid stream S_{C} obtained according to (i.2) or from the aqueous liquid stream S_{L} obtained according to (i.3) by evaporation in an evaporation unit E_{U} comprising least two evaporation sub-units E_{U1} and E_{U2}, obtaining at least one aqueous vapor stream S_{VE} and the aqueous liquid stream S_{R}.
29. The process of embodiment 28, wherein the solid material M provided according to (i.1.2) comprises, preferably consists of waste material, wherein said waste material preferably comprises textile waste material.
30. The process of embodiment 28 or 29 insofar as embodiments 28 or 29 are dependent on any one of embodiments 18 to 25, wherein according to (vii), passing the liquid stream S_{L2} to a downstream treatment stage comprises
   (vii.1) dividing the stream S_{L2} into a first stream S_{L21} and a second stream S_{L22}, wherein S_{L21} and S_{L22} have the same chemical composition as S_{L2};
   (vii.2) recycling the stream S_{L21} into the chemical reactor unit according to (i.1.5);
   (vii.3) passing the stream S_{L22} to a downstream treatment stage.
31. The process of embodiment 30, wherein the downstream treatment stage according to (vii.3) comprises one or more of
   a separation unit for separating at least one solid residue from the stream S_{L22};
   a processing unit for processing at least one solid residue comprised in the stream S_{L22};
   an incineration stage for incinerating at least one solid residue comprised in the stream S_{L22}.

In the context of the present invention, it is noted that the term “polyamide prepared from ε-caprolactam” as used herein refers to “polyamide 6” being characterized by the formula (—NH—(CH₂)₅—CO—)ₙ. In the context of the present invention, an ε-caprolactam monomeric compound CPM is an ε-caprolactam monomer. The term, “bar” as used in the context of the present invention refers to “bar(abs)”, i.e. bar (absolute), sometimes also referred to “bara”.

The term “textile material” covers textile raw materials and non-textile raw materials that are processed by various methods into linear, planar and spatial structures. It concerns the linear textile structures produced from them, such as yarns, twisted yarns and ropes, the sheet-like textile structures, such as woven fabrics, knitted fabrics, braids, stitch-bonded fabrics, nonwovens and felts, and the three-dimensional textile structures, i.e. body structures, such as textile hoses, stockings or textile semi-finished products; and it further concerns those finished products which, using the aforementioned products, are brought into a saleable condition by making up, opening up and/or other operations for onward transmission to the processor, the trade or the end consumer.

The term “textile waste material” covers a textile material as defined above, the inherent value of which has been consumed from the perspective of its current holder and, thus, is an end-of-life material for said holder.

In the context of the present invention, a term “X is one or more of A, B and C”, wherein X is a given feature and each of A, B and C stands for specific realization of said feature, is to be understood as disclosing that X is either A, or B, or C, or A and B, or A and C, or B and C, or A and B and C. In this regard, it is noted that the skilled person is capable of transfer to above abstract term to a concrete example, e.g. where X is a chemical element and A, B and C are concrete elements such as Li, Na, and K, or X is a temperature and A, B and C are concrete temperatures such as 10° C., 20° C., and 30° C. In this regard, it is further noted that the skilled person is capable of extending the above term to less specific realizations of said feature, e.g. “X is one or more of A and B” disclosing that X is either A, or B, or A and B, or to more specific realizations of said feature, e.g. “X is one or more of A, B, C and D”, disclosing that X is either A, or B, or C, or D, or A and B, or A and C, or A and, or B and C, or B and D, or C and D, or A and B and C, or A and B and, or B and C and D, or A and B and C and D.

### DESCRIPTION OF THE FIGURES

FIG. 1 is a schematic representation of a production unit used for the process according to preferred embodiments of the invention

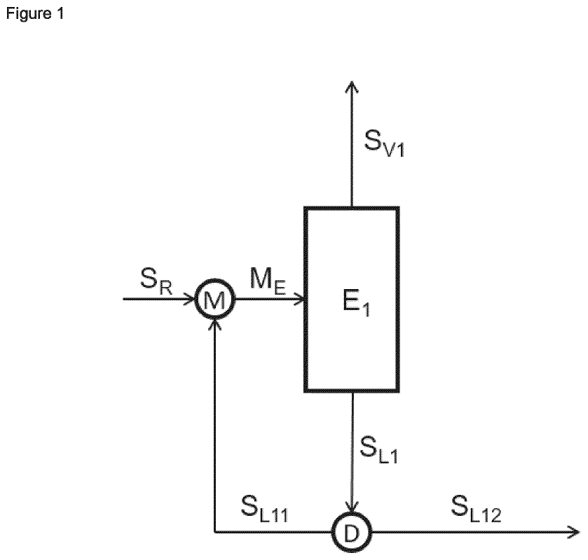

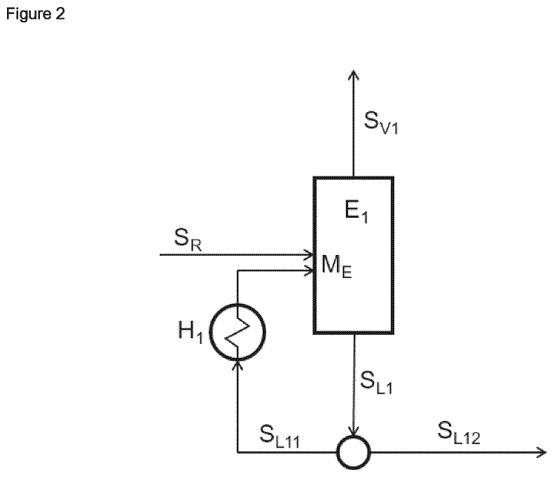

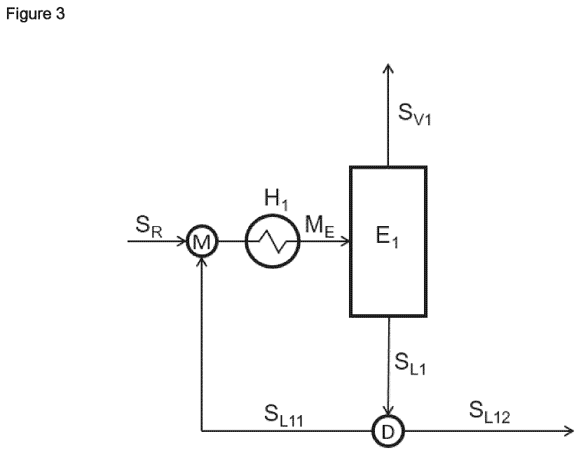

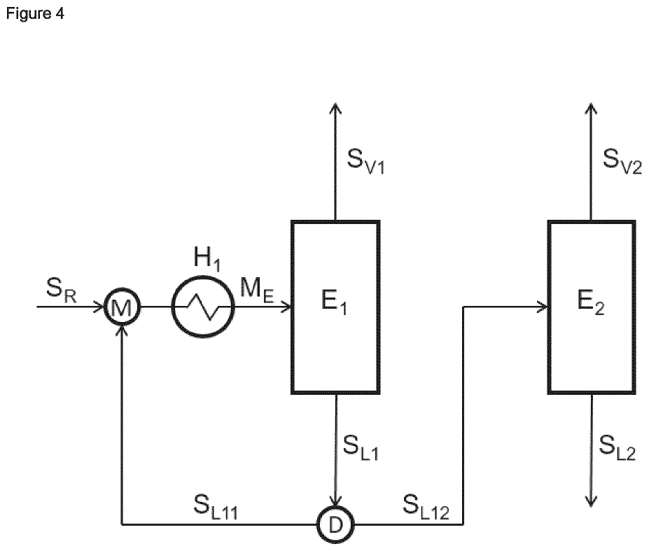

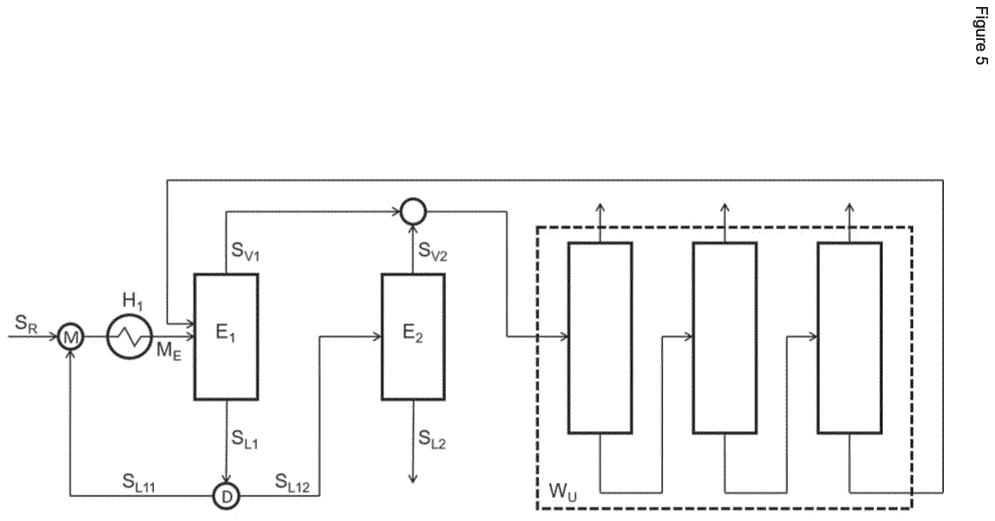

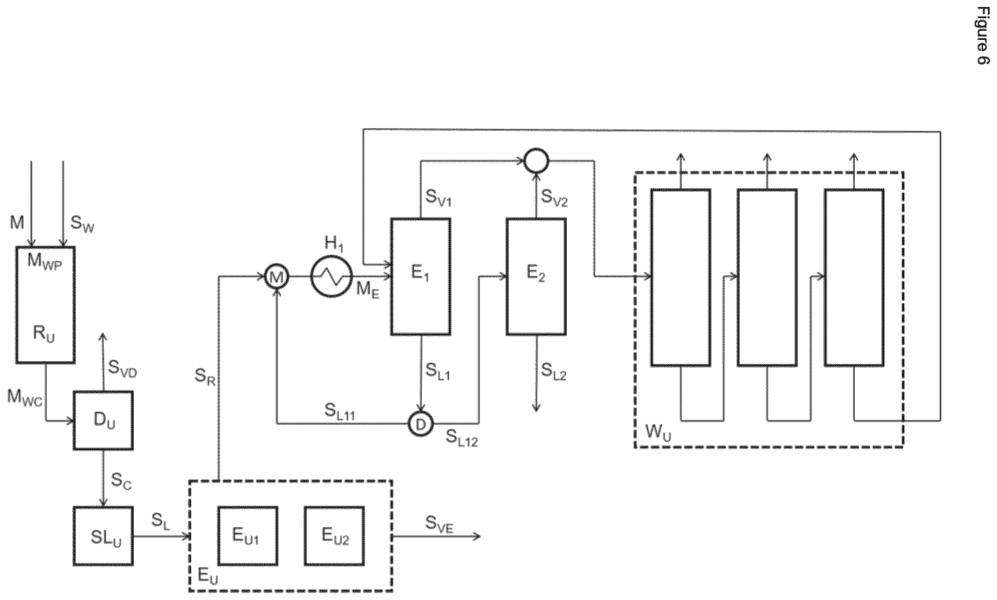

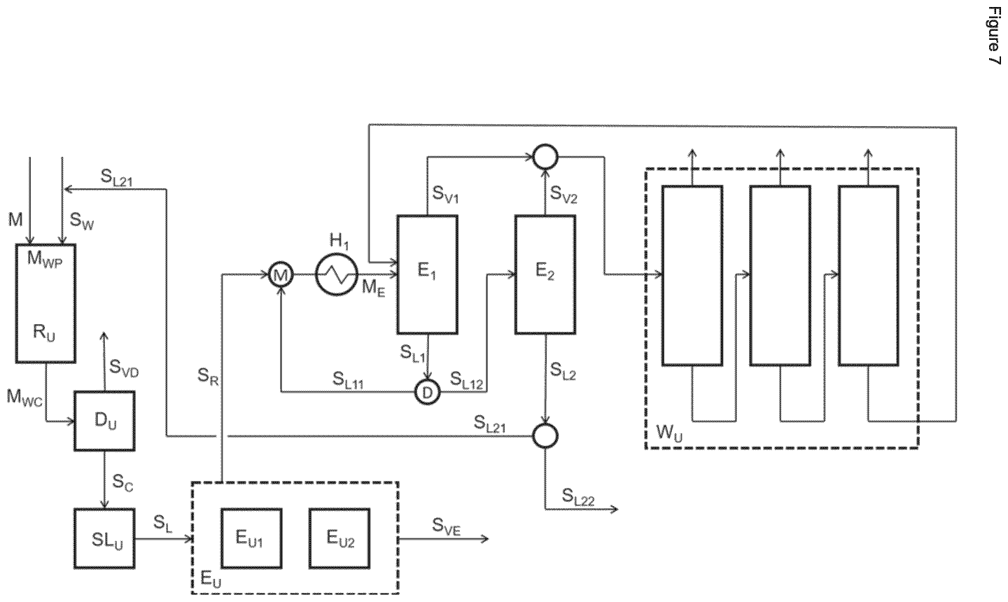

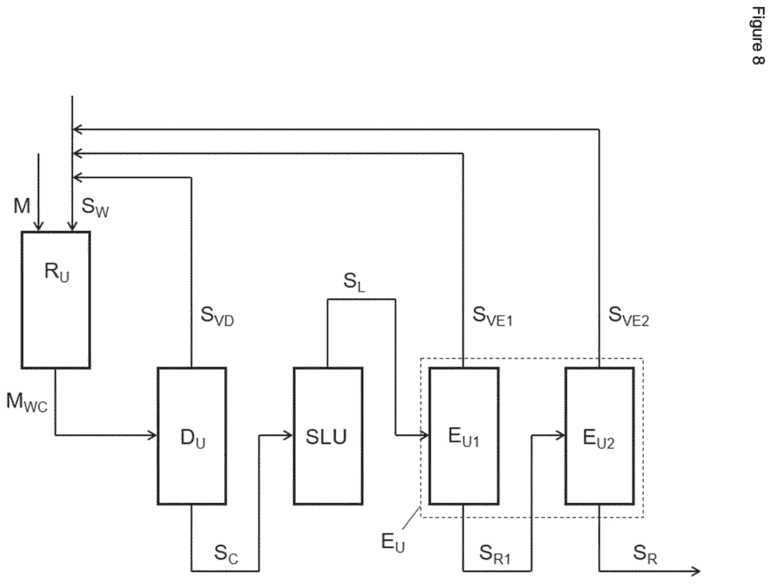

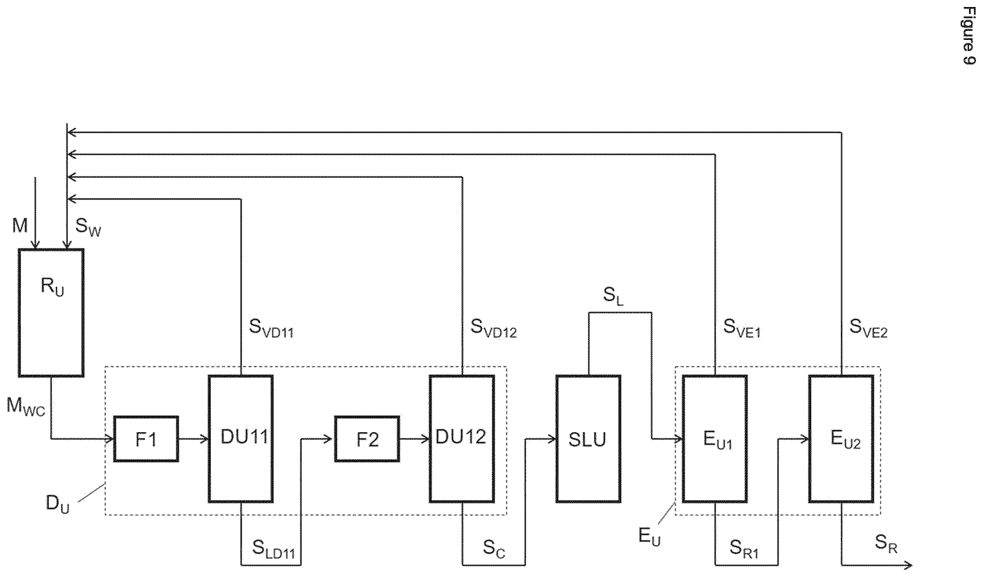

The production unit comprises an evaporation unit E₁, a dividing means D and a mixing means M. An aqueous liquid stream S_{R} comprising CPM dissolved in water at a concentration c_{R}(CPM), CPM having a boiling point T_{CPM}, wherein S_{R} further comprises the at least one CPO at a concentration c_{R}(CPO), CPO having a boiling point T_{CPO} with T_{CPO}>T_{CPM} is admixed with a stream S_{L11}, obtaining the aqueous liquid mixture M_{E}. The aqueous liquid mixture M_{E} is fed to evaporation conditions into the evaporation unit E₁, obtaining an aqueous vapor stream S_{V1} and an aqueous liquid stream S_{L1}. S_{V1} comprises CPM at a concentration c_{V1}(CPM) with c_{V1}(CPM)>c_{R}(CPM). S_{L1} comprises the at least one CPO at a concentration c_{L1}(CPO) with c_{L1}(CPO)>c_{R}(CPO) and comprises CPM at a concentration c_{L1}(CPM) with c_{L1}(CPM)<c_{R}(CPM). The aqueous liquid stream S_{L1} is divided in two streams S_{L11} and S_{L12}. S_{L11} and S_{L12} have the same chemical composition as S_{L1}. The aqueous liquid stream S_{L12} Is passed through a downstream treatment not shown in FIG. 1 and the aqueous liquid stream S_{L11} is recycled and mixed with SR.

FIG. 2 is a schematic representation of a production unit used for the process according to preferred embodiments of the invention

As in FIG. 1, the production unit comprises an evaporation unit E₁ and a dividing means D. However, compared to FIG. 1, the production unit further comprises a heat exchanger H₁ and does not comprise a mixing means M. The aqueous liquid stream S_{R} is fed into the evaporation unit E₁ and the aqueous liquid stream S_{L11}, prior to be recycled as a feed component into E₁, is passed through H₁ for heating. M_{E} is thus formed in E₁. Apart from said differences, the process illustrated in FIG. 2 is carried out as the one illustrated in FIG. 1.

FIG. 3 is a schematic representation of a production unit used for the process according to preferred embodiments of the invention

The process illustrated by FIG. 3 represents an alternative to the process illustrated by FIG. 2. As in FIG. 1, the production unit comprises an evaporation unit E₁, a dividing means D and a mixing means M. However, compared to FIG. 1, the production unit further comprises a heat exchanger H₁, said heat exchanger H₁ is positioned downstream of the mixing means M and upstream of E₁. Thus, H₁ heats the combined streams, preferably to a temperature in the range of from 200 to 270° C., more preferably in the range of from 210 to 270° C., more preferably in the range of from 220 to 270° C. Apart from said differences, the process illustrated in FIG. 3 is carried out as the one illustrated in FIG. 1.

FIG. 4 is a schematic representation of a production unit used for the process according to preferred embodiments of the invention

As in FIG. 3, the production unit comprises an evaporation unit E₁, a dividing means D, a mixing means M and a heat exchanger H₁. However, compared to FIG. 3, the production unit further comprises a second evaporation unit E₂. The aqueous liquid stream S_{L12} is fed to evaporation conditions in the second evaporation unit E₂, obtaining an aqueous vapor stream S_{V2} and a liquid stream S_{L2}. S_{V2} comprises CPM at a concentration c_{V2}(CPM) with c_{V2}(CPM)>c_{L12}(CPM), and CPO at a concentration c_{V2}(CPO) with c_{V2}(CPO)<c_{L12}(CPO). S_{L2} comprises the at least one CPO at a concentration c_{L2}(CPO) with c_{L2}(CPO)≥c_{L12}(CPO). The liquid stream S_{L2} is passed through a downstream treatment stage not shown in FIG. 4. Apart from said differences, the process illustrated in FIG. 4 is carried out as the one illustrated in FIG. 3.

FIG. 5 is a schematic representation of a production unit used for the process according to preferred embodiments of the invention

As in FIG. 4, the production unit comprises an evaporation unit E₁, a dividing means D, a mixing means M, a heat exchanger H₁ and a second evaporation unit E₂. However, compared to FIG. 4, the production unit further comprises a water removal unit W_{U} for separating CPM from water. The water removal unit W_{U} comprises 3 distillation columns serially arranged. The bottoms stream of the downstream-most distillation column is recycled into the evaporation unit E₁. The aqueous vapor stream S_{V1} and the aqueous vapor stream S_{V2} are mixed and the combined stream is passed through the water removal unit W_{U}. Apart from said differences, the process illustrated in FIG. 5 is carried out as the one illustrated in FIG. 4.

FIG. 6 is a schematic representation of a production unit used for the process according to preferred embodiments of the invention

As in FIG. 5, the production unit comprises an evaporation unit E₁, a dividing means D, a mixing means M, a heat exchanger H₁, a second evaporation unit E₂ and a water removal unit W_{U} for separating CPM from water. However, compared to FIG. 5, the production unit further comprises, upstream of M and E₁, a chemical reactor unit R_{U}, a depressurization unit D_{U}, a solid-liquid separation in a solid-liquid separation unit SL_{U} and an evaporation unit E_{U} comprising least two evaporation sub-units E_{U1} and E_{U2}. A solid material M containing a polyamide prepared from ε-caprolactam and an aqueous liquid stream S_{W} are fed to depolymerization conditions in a chemical reactor unit R_{U}, obtaining the aqueous liquid mixture M_{WC} comprising CPM dissolved in water and CPO. The aqueous liquid mixture M_{WC} is passed through a depressurization unit D_{U}, obtaining an aqueous vapor stream S_{VD} and an aqueous liquid stream S_{C}. S_{C} comprising CPM dissolved in water and CPO. The aqueous liquid stream S_{C} is subjected to solid-liquid separation by passing through the solid-liquid separation unit SL_{U}, obtaining an aqueous liquid stream S_{L} comprising CPM dissolved in water and CPO. Then, water is separated from aqueous liquid stream S_{L} by evaporation in E_{U} comprising E_{U1} and E_{U2}, obtaining an aqueous vapor stream S_{VE} and the aqueous liquid stream S_{R}. The aqueous liquid stream S_{R} is then treated as in the process illustrated in FIG. 5.

FIG. 7 is a schematic representation of a production unit used for the process according to preferred embodiments of the invention

As in FIG. 6, the production unit comprises an evaporation unit E₁, a dividing means D, a mixing means M, a heat exchanger H₁, a second evaporation unit E₂ and a water removal unit W_{U} for separating CPM from water and the production unit further comprises, upstream of M and E₁, a chemical reactor unit R_{U}, a depressurization unit D_{U}, a solid-liquid separation in a solid-liquid separation unit SL_{U} and an evaporation unit E_{U} comprising two evaporation sub-units E_{U1} and E_{U2}. The process illustrated in FIG. 7 is run as the process illustrated in FIG. 6 except that the liquid stream S_{L2} is divided in two streams, a first stream S_{L21} and a second stream S_{L22}. S_{L21} and S_{L22} have the same chemical composition as S_{L2}. The stream S_{L21} is recycled as a component of the aqueous liquid stream S_{W}. The liquid stream S_{L22} is passed through a downstream treatment stage not shown in FIG. 7.

FIG. 8 is a schematic representation of a portion of the production unit used for the process according to preferred embodiments of the invention, namely the portion which provides S_{R}

Said portion of the production unit comprises a reactor unit R_{U}, a depressurization unit D_{U}, a solid-liquid separation unit SL_{U} and an evaporation unit E₁ comprising two evaporation sub-units E_{U1} and E_{U2}, said two units are serially coupled as shown in FIG. 8. The solid material M comprising the polyamide and an aqueous liquid stream S_{W} are fed into the reactor unit R_{U} and subjected to depolymerization conditions comprising a depolymerization temperature T_{D} at a depolymerization pressure p_{D} as detailed in the foregoing. An aqueous liquid stream M_{WC} is removed from the bottom of R_{U}, M_{WC} comprising ε-caprolactam dissolved in water. The aqueous liquid stream M_{WC} is fed into the depressurization unit D_{U} obtaining an aqueous vapor stream S_{VD}, and an aqueous liquid stream S_{C} comprising ε-caprolactam dissolved in water. The aqueous vapor stream S_{VD} is recycled as a component of the aqueous liquid stream S_{W}, preferably after at least partial condensation. The aqueous liquid stream S_{C} is passed through the solid-liquid separation unit SL_{U} obtaining an aqueous liquid stream S_{SLU} comprising ε-caprolactam dissolved in water. The aqueous liquid stream S_{L} is then fed to evaporation in E₁, in particular S_{L} is fed to E_{U1}. An aqueous vapor stream S_{VE1} is obtained from E_{U1} and an aqueous vapor stream S_{VE2} is obtained from E_{U2}. The aqueous vapor streams S_{VE1} and S_{VE2} are recycled as a component of the aqueous liquid stream S_{W}, preferably via condensation. Further, an aqueous liquid stream S_{R1} comprising ε-caprolactam dissolved in water is removed from E_{U1} and fed into E_{U2} and an aqueous liquid stream S_{R} comprising ε-caprolactam dissolved in water is obtained and removed from E_{U2}. The aqueous liquid stream S_{R} is then further treated as disclosed in the foregoing and illustrated in FIGS. 1-7.

FIG. 9 is a schematic representation of a portion of the production unit used for the process according to preferred embodiments of the invention, namely the portion which provides S_{R}

Said portion of the production unit comprises a reactor unit R_{U}, a depressurization unit D_{U}, a solid-liquid separation unit SL_{U} and an evaporation unit E₁ comprising two evaporation sub-units E_{U1} and E_{U2}, said two units are serially coupled as shown in FIG. 9. The depressurization unit D_{U} comprises a depressurization sub-unit DU11, a depressurization sub-unit DU12 and two solid-liquid separation units F1 and F2. The aqueous liquid stream M_{WC} removed from the bottom of R_{U} is passed through the solid-liquid separation unit F1, preferably filtration unit F1, wherein F1 preferably has a mesh size in the range of from 0.5 to 5 mm, more preferably in the range of from 1 to 3 mm, prior to being fed into the sub-unit DU11 to obtain an aqueous vapor stream S_{VD11}, and an aqueous liquid stream S_{LD11} comprising ε-caprolactam dissolved in water. The aqueous liquid stream S_{LD11} is then passed through the solid-liquid separation unit F2, preferably filtration unit F2, wherein F2 preferably has a mesh size in the range of from 0.5 to 5 mm, more preferably in the range of from 1 to 3 mm, prior to being fed into the second depressurization sub-unit DU12, as a feed stream, to obtain an aqueous vapor stream S_{VD12}, and the aqueous liquid stream S_{C} comprising ε-caprolactam dissolved in water. The aqueous vapor streams S_{VD11} and S_{VD12} are recycled as a component of the aqueous liquid stream S_{W}, preferably after at least partial condensation. Downstream of D_{U}, the process is carried out as the process in FIG. 8.

1.-16. (canceled)

17. A process for separating at least one ε-caprolactam oligomeric compound CPO from a stream S_{R} comprising said at least one CPO and ε-caprolactam monomeric compound CPM, the process comprising
(i) providing an aqueous liquid stream S_{R} comprising CPM dissolved in water at a concentration c_{R}(CPM), CPM having a boiling point T_{CPM}, wherein S_{R} further comprises the at least one CPO at a concentration c_{R}(CPO), CPO having a boiling point T_{CPO} with T_{CPO}>T_{CPM};
(ii) preparing an aqueous liquid mixture M_{E} comprising the stream S_{R} provided according to (i);
(iii) subjecting the mixture M_{E} according to (i) to evaporation conditions in an evaporation unit E₁, obtaining an aqueous vapor stream S_{V1} and an aqueous liquid stream S_{L1}, wherein S_{V1} comprises CPM at a concentration c_{V1}(CPM) with c_{V1}(CPM)>c_{R}(CPM), wherein S_{L1} comprises the at least one CPO at a concentration c_{L1}(CPO) with c_{L1}(CPO)>c_{R}(CPO) and comprises CPM at a concentration c_{L1}(CPM) with c_{L1}(CPM)<c_{R}(CPM);
(iv) dividing the stream S_{L1} according to (iii) into a first stream S_{L11} and a second stream S_{L12}, wherein S_{L11} and S_{L12} have the same chemical composition as S_{L1};
(v) passing the stream S_{L12} obtained according to (iv) to a downstream treatment stage;
wherein preparing the aqueous liquid mixture M_{E} according to (ii) comprises mixing the stream S_{R} with the stream S_{L11}.

18. The process of claim 17,
wherein the stream S_{R} provided according to (i) exhibits a CPM concentration in the range of from 15 to 90 weight-%; and a CPO concentration in the range of from 0.5 to 10 weight-%;
wherein the stream S_{V1} obtained according to (iii) exhibits a CPM concentration in the range of from 65 to 99 weight-%; wherein the stream S_{V1} obtained according to (iii) preferably exhibits a CPO concentration in the range of from 0 to 0.4 weight-%;
wherein the stream S_{L1} obtained according to (iii) exhibits a CPM concentration in the range of from 0.1 to 10 weight-%; and a CPO concentration in the range of from 1 to 10 weight-%.

19. The process of claim 17, wherein preparing the aqueous liquid mixture M_{E} according to (ii) further comprises, prior to mixing the stream S_{R} with the stream S_{L11}, heating the stream S_{L11} obtained from (iv) to a temperature in the range of from 200 to 270° C.

20. The process of claim 19, wherein
heating the stream S_{L11} comprises passing the stream S_{L11} through a heat exchanger H₁; or wherein
preparing the aqueous liquid mixture M_{E} according to (ii) comprises mixing the stream S_{R} provided according to (i) with the stream S_{L11} obtained from (iv) and heating the combined stream to a temperature in the range of from 200 to 270° C., wherein heating the combined stream comprises passing the stream S_{L11} through a heat exchanger H₁.

21. The process of claim 17, wherein the evaporation in the evaporation unit E₁ according to (iii) is carried out in one or more stirred vessels, or in one or more film evaporators, or in one or more stirred vessels and in one or more film evaporators.

22. The process of claim 17, wherein the evaporation conditions according to (iii) comprise an evaporation temperature T_{E1} of the mixture M_{E}, wherein T_{E1} is in the range of from 200 to 270° C., and wherein the evaporation conditions according to (iii) further comprise an evaporation pressure p_{E1}.

23. The process of claim 17, wherein the evaporation conditions according to (iii) further comprise a residence time t_{E1} in the evaporation unit E₁, wherein t_{E1} is in the range of from 1 min to 5 h.

24. The process of claim 17, wherein according to (iv), the stream S_{L1} is divided into the first stream S_{L11} and the second stream S_{L12} at a mass ratio m(S_{L12}):m(S_{L11}) in the range of from 0.01:1 to 0.02:1.

25. The process of claim 17, wherein the downstream treatment stage according to (v) comprises one or more of
an evaporation unit;
a depolymerization unit for depolymerizing at least one of the at least one ε-caprolactam oligomeric compound CPO comprised in the stream SL12;
a separation unit for separating at least one solid residue from the stream S_{L12};
a processing unit for processing at least one solid residue comprised in the stream S_{L12};
an incineration stage for incinerating at least one solid residue comprised in the stream S_{L12}.

26. The process of claim 17, wherein the downstream treatment stage according to (v) comprises an evaporation unit, the process further comprising
(vi) subjecting the stream S_{L12} to evaporation conditions in an evaporation unit E₂, obtaining an aqueous vapor stream S_{V2} and a liquid stream S_{L2} wherein S_{V2} comprises CPM at a concentration c_{V2}(CPM) with c_{V2}(CPM)>c_{L12}(CPM), and CPO at a concentration c_{V2}(CPO) with c_{V2}(CPO)<c_{L12}(CPO); wherein S_{L2} comprises the at least one CPO at a concentration c_{L2}(CPO) with c_{L2}(CPO)≥c_{L12}(CPO);
(vii) passing the stream S_{L2} obtained according to (vi) to a downstream treatment stage.

27. The process of claim 26, wherein the evaporation in the evaporation unit E₂ according to (vi) is carried out in one or more stirred vessels, or in one or more film evaporators, or in one or more stirred vessels and in one or more film evaporators.

28. The process of claim 26, wherein the evaporation conditions according to (vi) comprise an evaporation temperature T_{E2} of the stream S_{L12}, wherein T_{E2} is in the range of from 200 to 300° C., and wherein the evaporation conditions according to (vi) further comprise an evaporation pressure p_{E2}.

29. The process of claim 26, wherein the evaporation conditions according to (vi) further comprise a residence time t_{E2} in the evaporation unit E₂, wherein t_{E2} is in the range of from 1 s to 5 min.

30. The process of claim 26, wherein the downstream treatment stage according to (vii) comprises one or more of
a depolymerization unit for depolymerizing at least one of the at least one ε-caprolactam oligomeric compound CPO comprised in the stream S_{L2};
a separation unit for separating at least one solid residue from the stream S_{L2};
a processing unit for processing at least one solid residue comprised in the stream S_{L2};
an incineration stage for incinerating at least one solid residue comprised in the stream S_{L2}.

31. The process of claim 17, the process further comprising
(viii) passing the aqueous vapor stream S_{V} to a water removal unit W_{U} for separating CPM from water.

32. The process of claim 17, wherein providing the stream S_{R} according to (i) comprises
(i.1) preparing an aqueous liquid mixture M_{WC} containing CPM dissolved in water and CPO, comprising
(i.1.1) providing an aqueous liquid stream S_{W};
(i.1.2) providing a solid material M containing a polyamide prepared from ε-caprolactam;
(i.1.3) preparing a mixture of the solid material M provided according to (i.1.2) and the aqueous liquid stream S_{W} provided according to (i.1.1);
(i.1.4) preparing an aqueous liquid mixture M_{WP} comprising the polyamide dissolved in water from the mixture prepared according to (i.1.3);
(i.1.5) subjecting the aqueous liquid mixture M_{WP} prepared according to (i.1.4) to depolymerization conditions in a chemical reactor unit R_{U}, obtaining the aqueous liquid mixture M_{WC} comprising CPM dissolved in water and CPO;
(i.2) optionally subjecting the aqueous mixture M_{WC} obtained according to (i.1.5) to depressurization in a depressurization unit D_{U}, obtaining an aqueous vapor stream S_{VD} and an aqueous liquid stream S_{C}, S_{C} comprising CPM dissolved in water and CPO;
(i.3) optionally passing the aqueous mixture M_{WC} obtained according to (i.1.5) or the aqueous liquid stream S_{C}, obtained according to (i.2) to solid-liquid separation in a solid-liquid separation unit SL_{U}, obtaining an aqueous liquid stream S_{L} comprising CPM dissolved in water and CPO;
(i.4) separating water from the aqueous liquid mixture M_{WC} obtained according to (i.1.5) or from the aqueous liquid stream S_{C} obtained according to (i.2) or from the aqueous liquid stream S_{L} obtained according to (i.3) by evaporation in an evaporation unit E_{U} comprising least two evaporation sub-units E_{U1} and E_{U2}, obtaining at least one aqueous vapor stream S_{VE} and the aqueous liquid stream S_{R}.
